(19) United States
(12) Patent Application Publication
(10) Pub. No.: US 2017/0095607 A1
(43) Pub. Date: Apr. 6, 2017

(54) Portable Syringe Storage and Disposal Device

(71) Applicant: MARK STEVEN WILSON, SANTA CRUZ, CA (US)

(72) Inventor: MARK STEVEN WILSON, SANTA CRUZ, CA (US)

(21) Appl. No.: 14/876,593

(22) Filed: Oct. 6, 2015

(51) Int. Cl. A61M 5/00; A61M 5/32

(52) U.S. Cl. CPC A61M 5/002; A61M 5/3205

(57) ABSTRACT

A device is provided for the storage, transportation and disposal of used medical syringes. The invented container provides a storage cavity for a used syringe, whereby the used syringe is preferably depressed into the storage cavity such that the top of the used syringe is substantively below a projection set, such that the used syringe may not be easily removed from the storage cavity. The invented container may further create a friction fit around the used syringe, providing an additional layer of redundancy for the prevention of the removal of the used syringe from the storage cavity. The storage cavity may be stand-alone within container, or one container may optionally contain a plurality of storage cavities. The invented container may further provide a storage area for an unused syringe, whereby the unused syringe may be removed from the container with relative facility by a user. The storage area for the used syringe and the unused syringe may optionally be the same storage area within the invented container.

### FIELD OF THE INVENTION

The present invention relates to methods and devices intended to enable the safe disposal of syringes. More particularly, the present invention relates to systems and methods by which used syringes may be safely discarded.

### BACKGROUND OF THE INVENTION

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also be inventions.

Outside of medical facilities, syringes are primarily used by two categories of individuals: diabetics and unauthorized users of various drugs and opiates. In both of these categories, a means by which used syringes may be easily transported and disposed of provides both a hygienic necessity, and a greater convenience. Particularly necessary is a means by which used syringes may be prevented from continued use. This prevention of re-use of syringes helps to minimize the spread of transmissible diseases, particularly among users of various drugs and opiates, who might otherwise share and/or re-use unsanitary syringes.

In the prior art, stand-alone disposal stations for used syringes are frequently inconvenient to those using the syringes, and thus the used syringes are disposed of in manners not conducive to individual or public health. The prior art fails, however, to optimally provide for a means by which a syringe may be securely inserted into a cavity from which the syringe may not be subsequently removed.

There is therefore a long-felt need to provide a secure, post-use storage container for syringes.

### SUMMARY AND OBJECTS OF THE INVENTION

Towards these objects and other objects that will be made obvious in light of the present disclosure, a device is provided by which medical syringes may be contained after use.

In a first preferred embodiment of the present invention, a cavity is provided into which a used syringe may be placed to be securely stored for disposal. The cavity preferably includes a projection set underneath which the used syringe may be pushed, so that the top of the plunger of the syringe may form a hermetic or near-hermetic seal with the projection set. Additionally presented is a projection set which may optionally cover the finger grip of the syringe, providing an additional seal against both the removal of the used syringe from the device, and the escape of liquid from a leak in the needle of the syringe. The projection set by which the syringe may be stabilized may optionally be one or more individual projections from a rim of the cavity, or may optionally be a full ring, encircling the entirety of the rim of the cavity, or a partial ring.

Some or all of the length of the cavity may have a geometric cross-sectional shape of a circle, polygon, quadrilateral, or square. In addition, the internal cavity may have two sections, wherein the section comprising the projection sets has one shape, e.g. circular or polygonal, and the lower portion of the cavity may have an alternate shape, e.g. circular or polygonal.

In a further preferred embodiment of the present invention, a syringe is provided having a plunger and a finger grip by which the syringe may be stabilized for use by a user. The diameter of the finger grip of the syringe is preferably equal to or less than the substantively parallel diameter of the plunger top of the syringe.

In an additional preferred embodiment of the present invention, a storage container is provided having a plurality of the above-described cavities, wherein a plurality of syringes may be stored conveniently for a user.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE FIGURES

These, and further features of the invention, may be better understood with reference to the accompanying specification and drawings depicting the preferred embodiment, in which:

FIG. 1A is a side view of an invented syringe;

FIG. 1B is a rotated top view of the invented syringe;

FIG. 2A is a side view of a first invented cavity with no syringe therein;

FIG. 2B is a top view of the first invented cavity with no syringe therein;

FIG. 2C is a side view of the first invented cavity containing the invented syringe;

FIG. 3A is a side view of a second embodiment of the invented cavity while empty, including a bottom of the cavity having a smaller width than a top of the cavity;

FIG. 3B is a top view of the second embodiment of the invented cavity while empty, including the bottom of the cavity having a smaller width than the top of the cavity;

FIG. 3C is a side view of the second embodiment of the invented cavity containing the invented syringe, including the bottom of the cavity having a smaller width than the top of the cavity;

FIG. 4 is a cut-away side view of the invented storage device showing a plurality of cavities in which used and unused syringes may be securely stored;

FIG. 5 is a side view of the invented storage device showing a plurality of cavities in which used syringes may be stored and a second, separate plurality of cavities in which unused syringes may be stored;

FIG. 6A is a cut-away side view of an alternate embodiment of the invented storage device having a single cavity, and containing an unused syringe;

FIG. 6B shows a plurality of the invented storage device of FIG. 6A contained within a generic storage container;

FIGS. 7A-7D are top views of a plurality of preferred embodiments of the projection sets of the invented cavity; and

FIGS. 8A-8E are cut-away side views of a plurality of design variations for the invented cavity.

### DETAILED DESCRIPTION

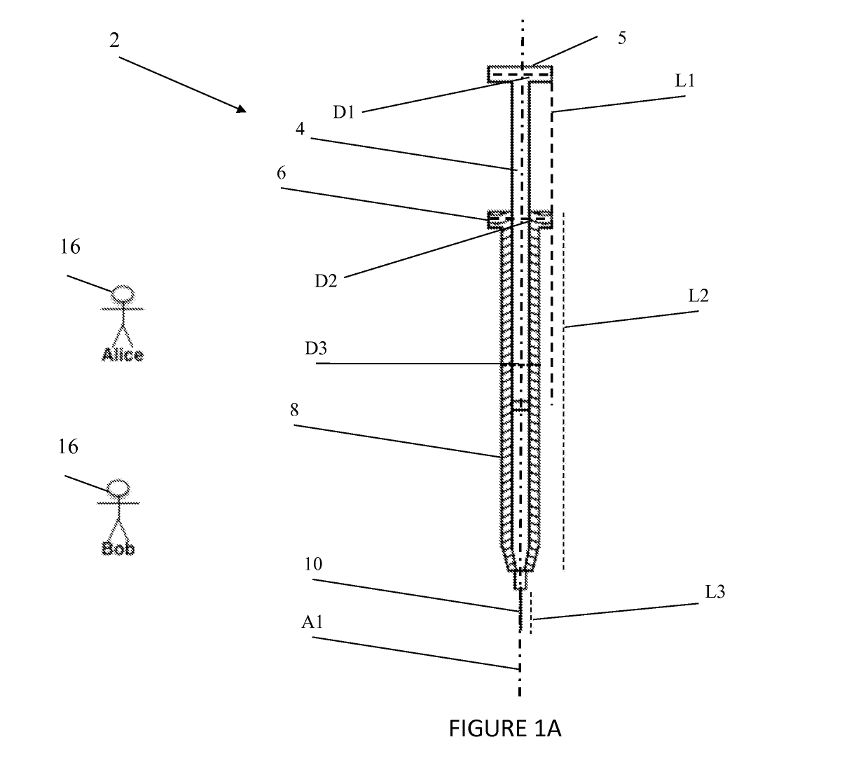

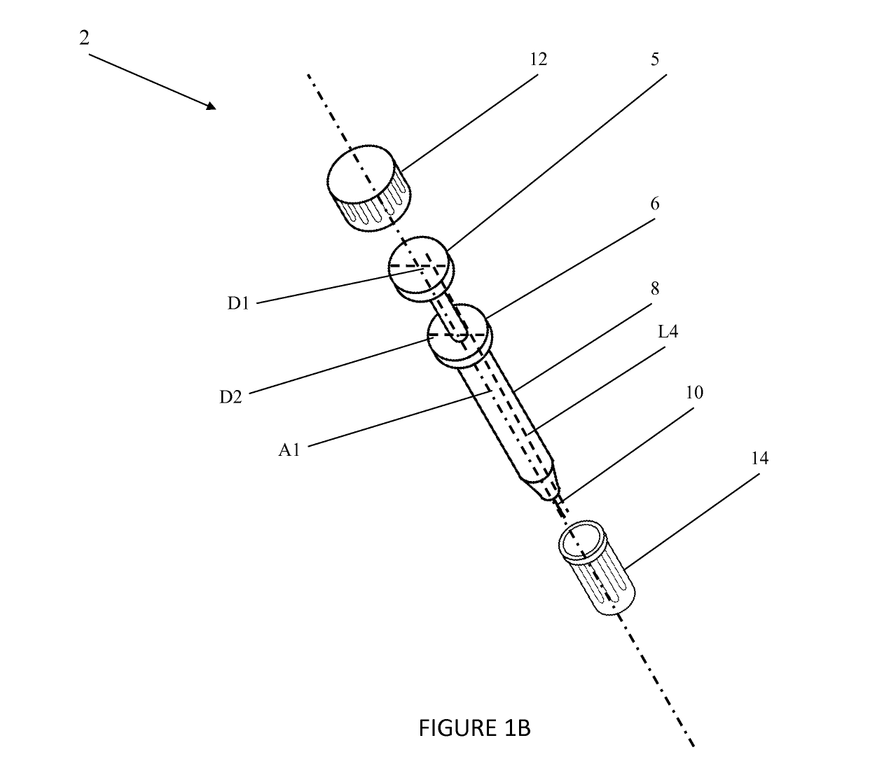

Referring now generally to the Figures and particularly to FIG. 1A, FIG. 1A is a side view of a syringe 2, wherein the syringe 2 comprises a plunger 4 having a plunger top 5, a finger grip 6, a barrel 8, and a needle 10. The diameter D1 of the plunger top 5 and the diameter D2 of the finger grip 6 are preferably substantively identical, or the diameter D2 of the finger grip 6 may optionally be less than the diameter D1 of the plunger top 5, but is preferably not greater than the diameter D1 of the plunger top 5. The diameter D3 of the barrel 8 of the syringe 2 is preferably less than the diameter D1 of the plunger top 5 and/or of the diameter D2 of the finger grip 6. Also presented is a length L1 of the plunger top 5, a length L2 of the barrel 6 of the syringe 2, and a length L3 of the needle 10.

The length L1 of the plunger, the length L2 of the barrel and the length L3 of the needle each preferably extend along a first elongate syringe axis A1.

The elements of the syringe 2 are preferably substantively circular, thus the inclusion of the diameters D1, D2 and D3 of the plunger top 5, the finger grip 6 and the barrel 8, respectively, but may optionally or alternatively have a polygonal or quadrilateral cross-sectional shape. The diameter D1 of the plunger top 5, the diameter D2 of the finger grip 6, and the diameter D3 of the barrel 8 each are preferably orthogonal to the first elongate syringe axis A1.

The plunger top 5, finger grip 6 and the barrel 8 may optionally be composed of flexible materials, including but not limited to elastomers, or may be composed of inflexible materials, such as non-rubberized plastic, glass and/or metal, or other suitable syringe creation materials known in the art.

Referring now generally to the Figures and particularly to FIG. 1B, FIG. 1B is a rotated top view of the syringe 2, including the elements described above in FIG. 1A, and further including a plunger cap 12 and a needle cap 14. The plunger cap 12 and the needle cap 14 serve to protect the plunger top 5 and the needle 10, respectively, while the syringe 2 is in a storage position, prior to use. The plunger cap 12 and the needle cap 14 further create a quick and easy notification to a user 16, nominally Alice 16 or Bob 16, that the syringe 2 has not yet been used. The plunger cap 12 and the needle cap 14 are preferably composed of hard plastic materials, though may composed of other suitable materials known in the art. A syringe length L4 is shown to extend along the entire length of the syringe 2 from the plunger top 5 to the bottom of the needle 10, along the first elongate syringe axis A1.

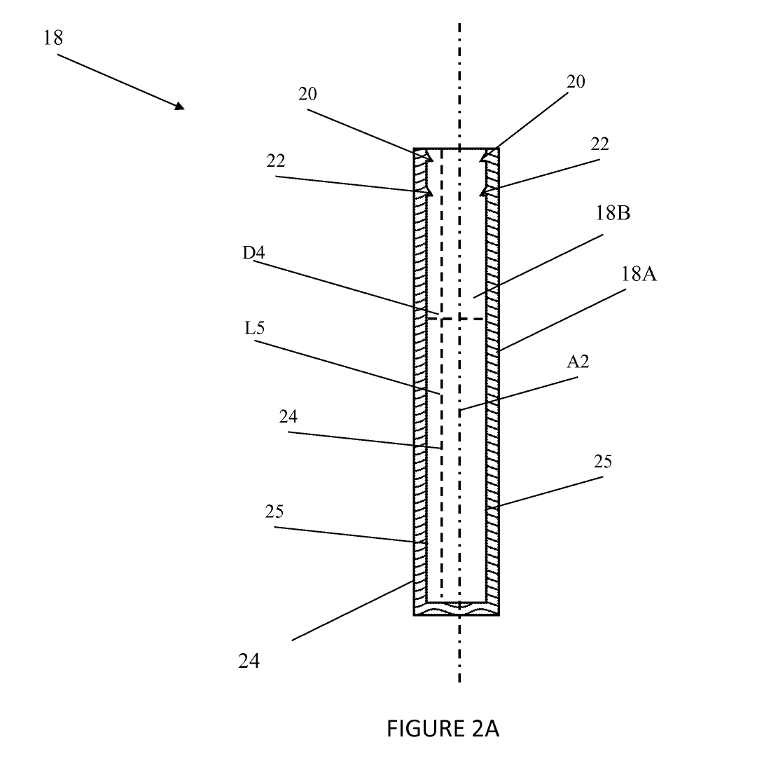

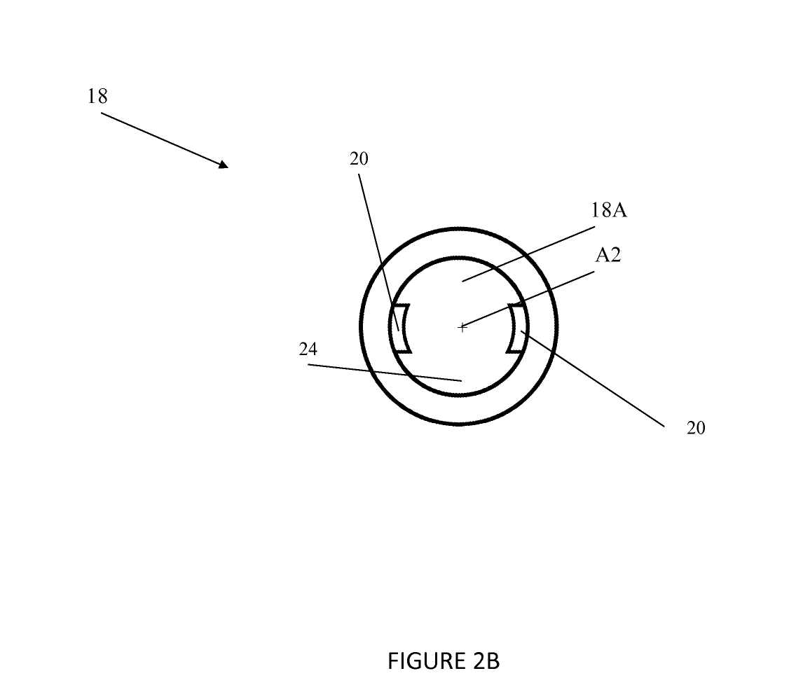

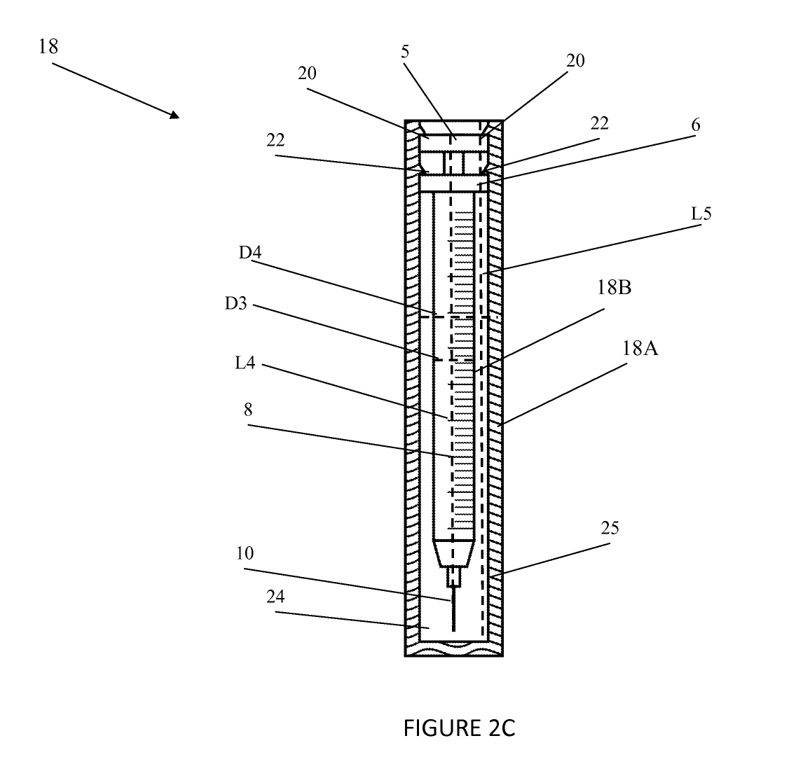

Referring now generally to the Figures, and particularly to FIG. 2A, FIG. 2A is a cut-away side view of a first invented elongate device 18 (hereinafter “first invented device 18”), including a first body 18A that forms a first cavity 18B. The first invented device 18 includes a plunger projection set 20, wherein the plunger projection set 20 substantively prevents the plunger 4 from escaping from the first invented device 18 when the plunger top 5 is inserted substantively fully into the first invented device 18 when the syringe 2 has been used, and preferably provides a hermetic or near-hermetic seal when coupled with the plunger top 5, preventing any escape of liquid from the first invented device 18. The first invented device 18 further preferably, but not necessarily, includes a finger grip projection set 22, wherein the finger grip projection set 22 substantively prevents the finger grip 6 from moving within the first invented device 18, or from escaping from the first invented device 18 when the finger grip 6 has been inserted into the first invented device 18, and pushed below the finger grip projection set 22. The plunger projection set 20 preferably protrudes toward a first elongate device axis A2 of the first invented device 18 in a range of ratios of the diameter D1 of the plunger top 5. More particularly, the plunger projection set 20 preferably protrudes toward the first elongate device axis A2 of the first invented device 18 in a range of 0.1-0.5 of the diameter D1 of the plunger top 5. Yet more particularly, the plunger projection set 20 preferably protrudes toward the first elongate device axis A2 of the first invented device 18 at a ratio of 0.2-0.4 of the diameter D1 of the plunger top 5. The finger grip projection set 22, when present, preferably also protrudes toward the first elongate device axis A2 of the first invented device 18 in a rage of ratios of the diameter D2 of the finger grip 6. More particularly, the optional finger grip projection set preferably protrudes toward first elongate device axis A2 of the first invented device 18 in a range of 0.1-0.5 of the diameter D2 of the finger grip 6. Yet more particularly, the finger grip projection set 22 preferably protrudes toward the first elongate device axis A2 of the first invented device 18 at a ratio of 0.2-0.4 of the diameter D2 of the finger grip 6.

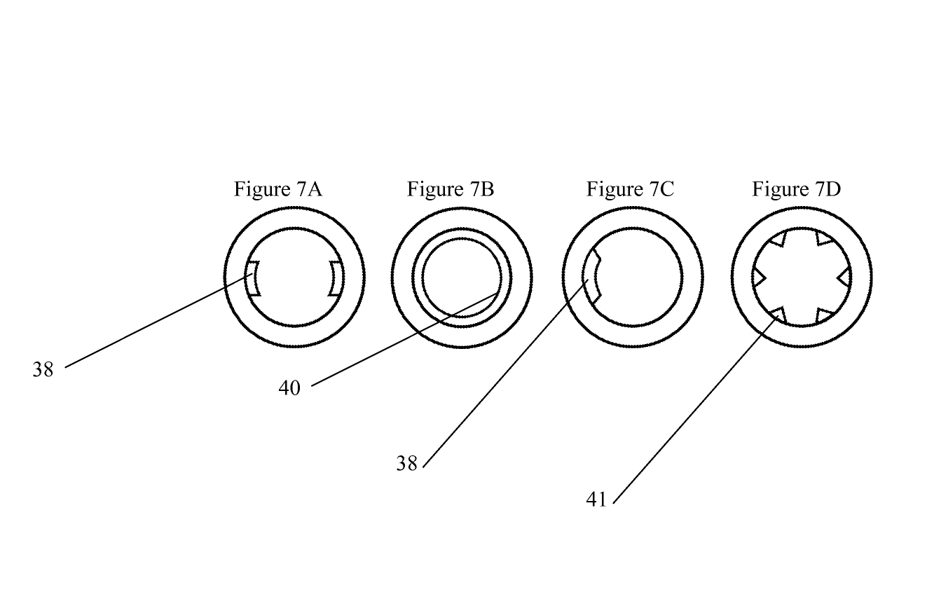

The first body 18A forms a first well 24 and one or more first internal walls 25, into which the barrel 8 and needle 10 of the syringe 2 may be inserted, preferably following the use of the syringe 2. The first invented device 18 has a first device length L5 that extends along the first elongate device axis A2, wherein the first device length L5 along the first elongate device axis A2 is preferably greater than the syringe length L4 along the syringe elongate axis A1. A first cavity diameter D4 is preferably set orthogonally to the first elongate device axis A2. The plunger projection set 20 and the finger grip projection set 22 may optionally be one or more of several types of projection, as discussed in more detail below, in reference to FIGS. 7A-7D.

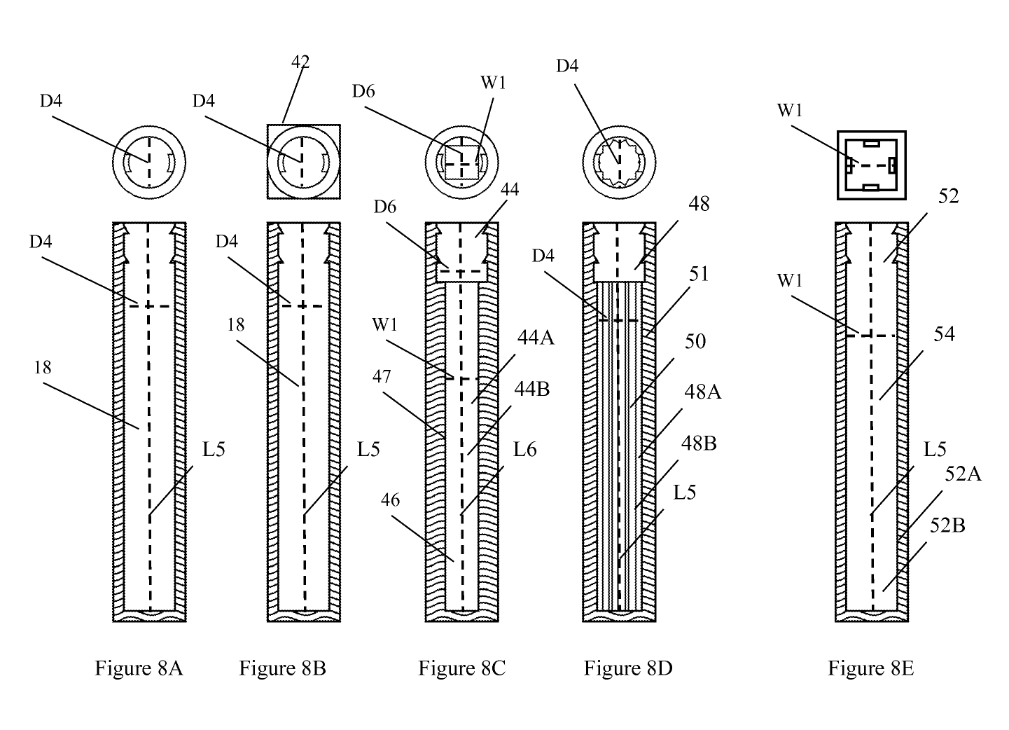

The diameter D4 of the first well 24 of the first invented device 18 is preferably, though not necessarily, slightly larger than the diameter D3 of the barrel 8 of the syringe 2, such that the primary means by which the syringe 2 is held in place within the first invented device 18 is the plunger projection set 20, and optionally the finger grip projection set 22. The first well 24 of the first invented device 18 is shown in the Figure to be cylindrical, but may optionally be a plurality of cross-sectional shapes, including, but not limited to, quadrilateral or polygonal as discussed in more detail below, in reference to FIGS. 8A-8E.

The first invented device 18 forms a non-porous first body 18A and may optionally be comprised of or including of any suitable water-resistant, non-porous materials known in the art, including, but not limited to polyethylene, polyurethane, or polypropylene, but may be any other suitable material known in the art.

Referring now generally to the Figures and particularly to FIG. 2B, FIG. 2B is a top view of the first invented device 18, showing the plunger projection set 20 extending orthogonally from the internal wall 25 and toward the first elongate device axis A2. The plunger projection set 20 is shown in the Figure to be two partial and separate projections, but this is exemplary embodiment not intended as a limitation as to the possible variety of shapes for the plunger projection set 20, but only as an illustration as one possible means by which the plunger 4 may be contained within the first invented device 18, as discussed in more detail below, in reference to FIGS. 7A-7D.

Referring now generally to the Figures and particularly to FIG. 2C, FIG. 2C is a cut-away side view of the first invented device 18, wherein the first invented device 18 contains a syringe 2. The plunger projection set 20 is shown to substantively inhibit the plunger 4 from exiting or being removed from within the first cavity 18B of the first invented device 18. When the syringe 2 is inserted fully into the first invented device 18, the first body 18A in combination with the plunger projection set 20 and the plunger top 5, create a hermetic or near-hermetic seal, such that little or no liquid which may leak out of the needle 10 may escape from the first invented device 18 from the volume defined between the well 24 to below the position of the plunger top 5.

The optional finger grip projection set 22 in combination with the syringe 2 may also create a hermetic or near-hermetic seal within the first invented device 18 when the finger grip 6 of the syringe 2 is inserted fully into the first invented device 18 below the finger grip projection set 22 to create an additional sealing layer to prevents or reduces liquid leaks from a second volume defined within the first cavity 18B, wherein the second volume is defined by the first body 18A between the well 24 and the finger grip 22 when the finger grip 22 is positioned below the optional finger grip projection set 22. Furthermore, the first invented device 18 is preferably composed of a non-porous, liquid-proof material, so that any leaks from the needle 10 may be substantively contained within the first cavity 18B of the first invented device 18.

In the Figure, the diameter D4 of the first invented device 18 is shown to be sufficiently greater than the diameter D3 of the barrel 8 of the syringe 2 so that the first internal walls 25 of the barrel 8 of the syringe 2 does not touch the first internal walls 25 of the well 24 of the first invented device 18. This is for illustrative purposes only, and should not be construed as an exclusion on the size of the well 24 of the first invented device 18. In the current embodiment of the first invented device 18, the first internal walls 25 of the well 24 may optionally be close enough to the barrel 8 of the syringe 2 to touch the barrel 8, or may be separated, as shown. The diameter D1 of the plunger top 5 and the diameter D2 of the finger grip 6 are preferably substantively equal to the diameter D4 of the first invented device 18, such that a hermetic or near-hermetic seal may be created and maintained by the insertion of the finger grip 6 and the plunger top 5 into the first invented device 18.

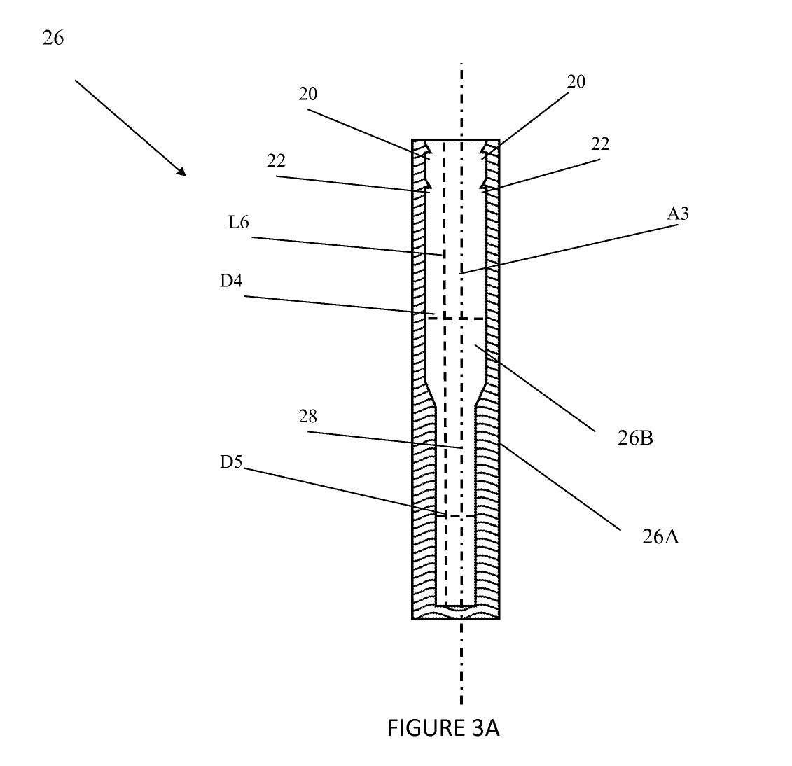

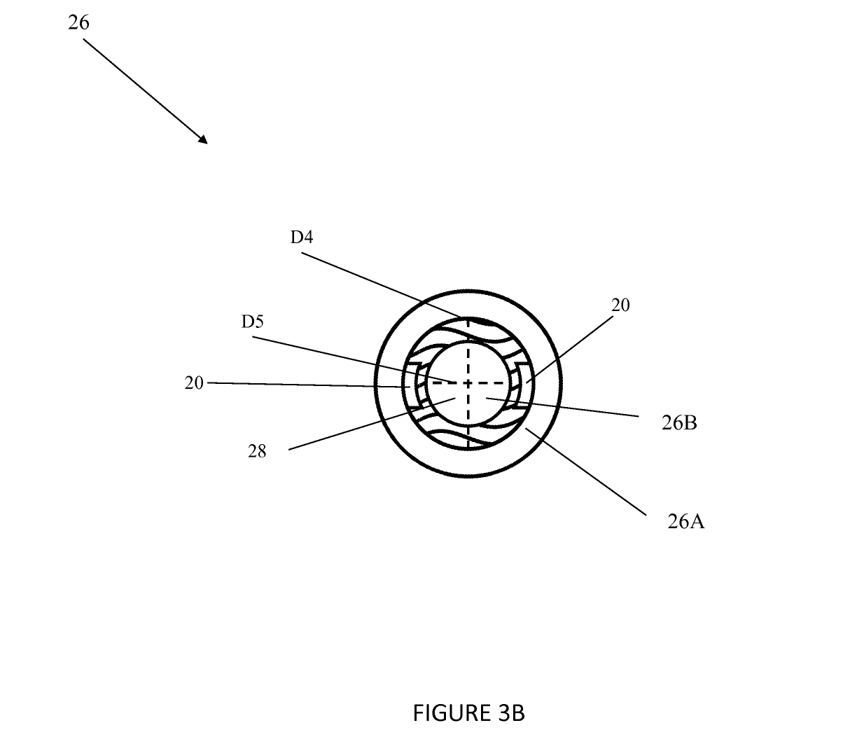

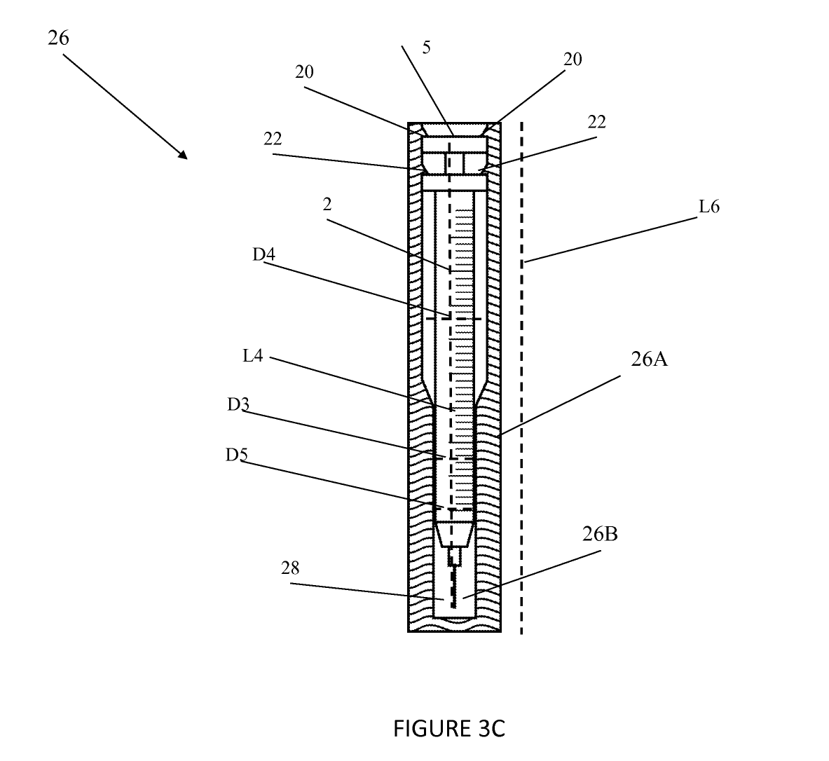

Referring now generally to the Figures, and particularly to FIG. 3A, FIG. 3A is a cut-away side view of a second embodiment of the invented elongate device 26 (hereinafter “second invented device” 26). The second invented device 26 comprises a second body 26A having the plunger projection set 20, optionally the finger grip projection set 22, a second well 28 and one or more second internal walls 29 forming a second cavity 26B. The second invented device 26 further presents a second device elongate axis A3. The portion of the second well 28 along the second device elongate axis A3 nearer to the plunger projection set 20 and the optional finger grip projection set 22 preferably has the diameter D4 of the well 24 of the first invented device 18; the diameter D4 of the first invented device 18, when present in the second invented device 26, is preferably set orthogonally to the second device elongate axis A3. The portion of the second well 28 having a greater distance from the plunger projection set 20 and the finger grip projection set 22 has a diameter D5 which represents some fraction of the diameter D4 of the upper portion of the well 28. The fraction of the diameter D4 of upper portion of the well 28 that the lower portion diameter D5 represents may preferably, optionally be within the range of 0.25-0.75 of the diameter of the diameter D4 of the upper portion of the well 28.

The portion of the second well 28 along the second device elongate axis A3 that is closer to the plunger projection set 20 and the finger grip projection set 22 preferably spans approximately half of a second device length L6 along second device elongate axis A3, but may optionally comprise more or less than half of the second device length L6. The portion of the second well 28 at the cavity length L6 having a greater distance from the plunger projection set 20 and the finger grip projection set 22 preferably spans approximately half of the second device length L6 along second device elongate axis A3, but may optionally comprise more or less than half of the second device length L6.

The second internal walls 29 of the second well 28 may optionally be composed of a flexible material whereby the barrel 8 of the syringe 2 may displace the flexible material for the purpose of ensuring the friction fit or interference fit between the barrel 8 of the syringe 2 and the material of the second internal walls 29 of the second well 28. Alternately, the second internal walls 29 of the second well 28 may be composed of an inflexible material, whereby the snug fit of the barrel 8 within the second well 28 may be sufficient to maintain a friction fit or interference fit therebetween. The material for the second well 28 is preferably non-porous and liquid resistant, such that any leak from the tip of the needle 10 may be contained within the second well 28 by means of both the fit between the barrel 8 and the walls 29, and by means of the hermetic or near-hermetic seal between the plunger top 5 and the plunger projection set 20. The non-porous second body 26A of the second invented device may optionally be comprised of or including any suitable water-resistant, non-porous materials known in the art, including, but not limited to polyethylene, polyurethane, or polypropylene, but may be any other suitable material known in the art.

Referring now generally to the Figures, and particularly to FIG. 3B, FIG. 3B is a top view of the second invented device 26, showing the plunger projection set 20, and the upper diameter D4 and the lower diameter D5 of the second well 28. The Figure shows the difference in diameter between the upper portion and lower portions of the second well 28.

Referring now generally to the Figures, and particularly to FIG. 3C, FIG. 3C is a cut-away side view of the second invented device 26, having a syringe 2 stored therein. The diameter D5 of the lower portion of the second well 28 creates a friction fit or interference fit with the lower portion of the barrel 8 of the syringe 2. The diameter D5 of the lower portion of the second well 28 is preferably, but not necessarily, within the range of 0.005 inch-0.025 inch of the diameter D3 of the barrel 8 of the syringe 2. More particularly the diameter D5 of the lower portion of the second well 28 is preferably, but not necessarily, within the range of 0.010 inch-0.020 inch of the diameter D3 of the barrel 8 of the syringe 2. Yet more particularly, the diameter D5 of the lower portion of the second well 28 is preferably, but not necessarily, within 0.015 inch of the diameter D3 of the barrel 8 of the syringe 2. The tightness of the lower portion of the second well 28 provides security against movement of the syringe 2, in addition to the hermetic or near-hermetic seal provided by the coincidence of the plunger projection set 20 and the plunger top 5 of the syringe 2, and optionally of the finger grip projection set 22 and the finger grip 6 of the syringe 2. Additionally shown is that the entire length L4 of the syringe 2 along the first elongate syringe axis A1 may be contained within the second device length L6 along the second device elongate axis A3 of the second invented device 26.

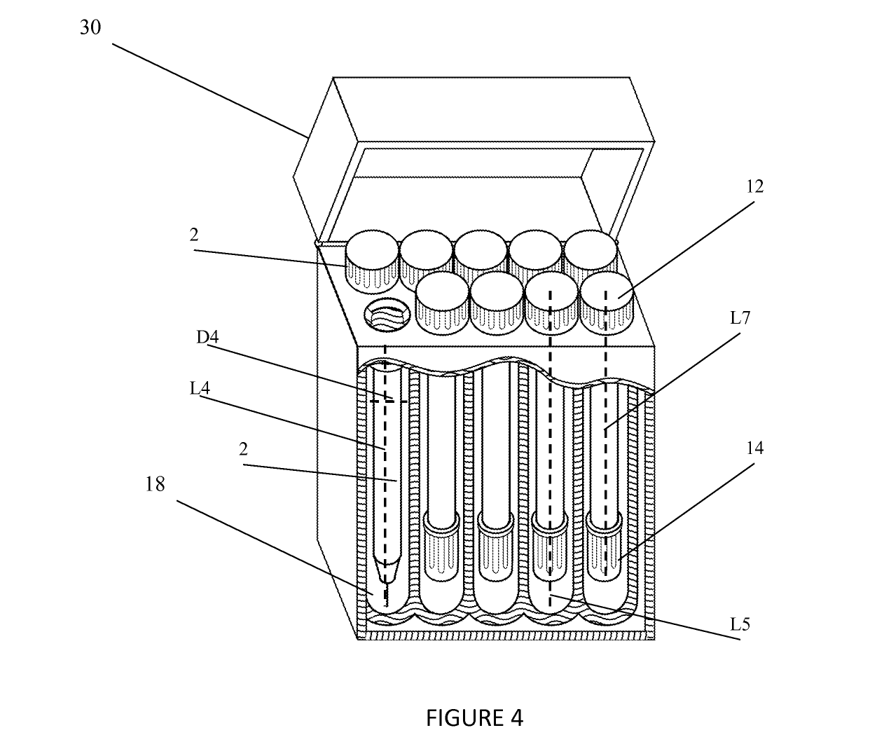

Referring now generally to the Figures and particularly to FIG. 4, FIG. 4 is a cut-away view of a first invented container 30 in which used and unused syringes 2 may be stored in the same bodies. The first container 30 houses a plurality of the first invented devices 18 or the second invented devices 26, wherein an unused syringe 2 preferably, but not necessarily, having a plunger cap 12 and a needle cap 14 placed thereon, and having an unused syringe length L7, may be accessibly stored prior to use. The plunger cap 12 of the unused syringe 2 optionally allows the syringe 2 to remain accessibly secured, rather than fully secured, within the first invented device 18 or the second invented device 26 by resting on the top of the first invented device 18 or the second invented device 26. Once the syringe 2 has been used by the user 16, the syringe 2 may be returned to the first invented device 18 or the second invented device 26 from which it was taken, and hermetically or near-hermetically sealed within the first invented device 18 or the second invented device 26 by means of the plunger projection set 20 and optionally by the finger grip projection set 22, and further optionally, in the case of the second invented device 26, by means of a friction fit or interference fit between the barrel 8 of the syringe 2 and the second internal walls 29 of the second well 28. The syringe length L4 and the unused syringe length L7, each along the syringe elongate axis A1, are both preferably less than the first device length L5. The diameter D4 of the first invented device 18 and diameter D4 and diameter D5 of the second invented device 26 are preferably great enough to house both the used and the unused syringes 2. Additionally, the diameter D4 of the first invented cavity within the first container 30 is preferably set orthogonally to the first elongate device axis A2, and the diameter D4 and the diameter D5 of the second invented device 26 within the container 30 are preferably set orthogonally to the second elongate cavity axis A3.

The first container 30 preferably has a weight and size convenient to the user 16, and is preferably, but not necessarily, approximately the dimensions and weight of a cigarette box. The materials used to create the first container 30 are preferably polyethylene, polyurethane, or polypropylene, but may be any other suitable material known in the art.

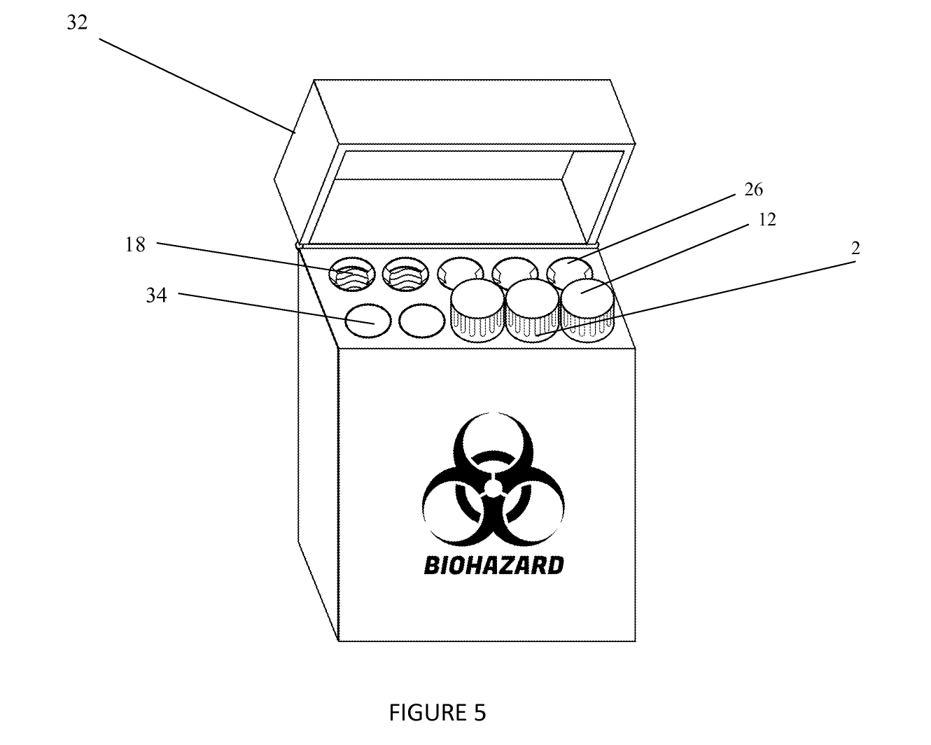

Referring now generally to the Figures and particularly to FIG. 5, FIG. 5 is a side view of a second container 32. The second container 32 includes a plurality of first invented devices 18 and/or second invented devices 26 for the storage of syringes 2 that have been used by the user 16. The second container 32 further includes a plurality (preferably the same number as the plurality of first invented devices 18 and/or second invented devices 26) of slots 34 into which syringes 2 which have not been used may be removably accessed by the user 16. The plunger cap 12 of the syringe 2 may serve as a means by which the syringe 2 may remain accessible to the user 16 within the second container 32.

The second container 32 preferably has a weight and size convenient to the user 16, and is preferably, but not necessarily, approximately the dimensions and weight of a cigarette box. The materials used to create the second container 32 are preferably polyethylene, polyurethane, or polypropylene, but may be any other suitable material known in the art.

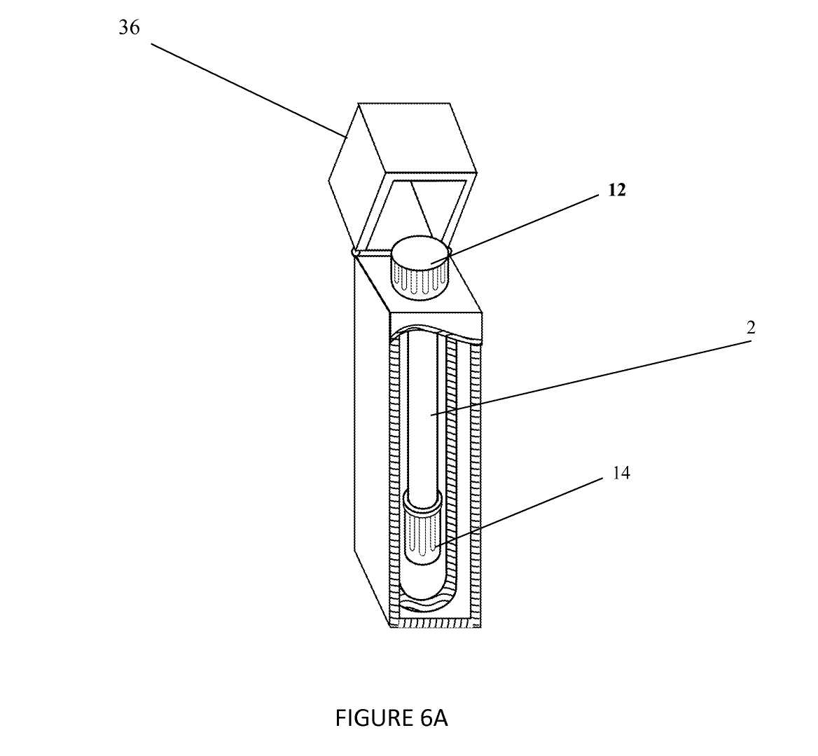

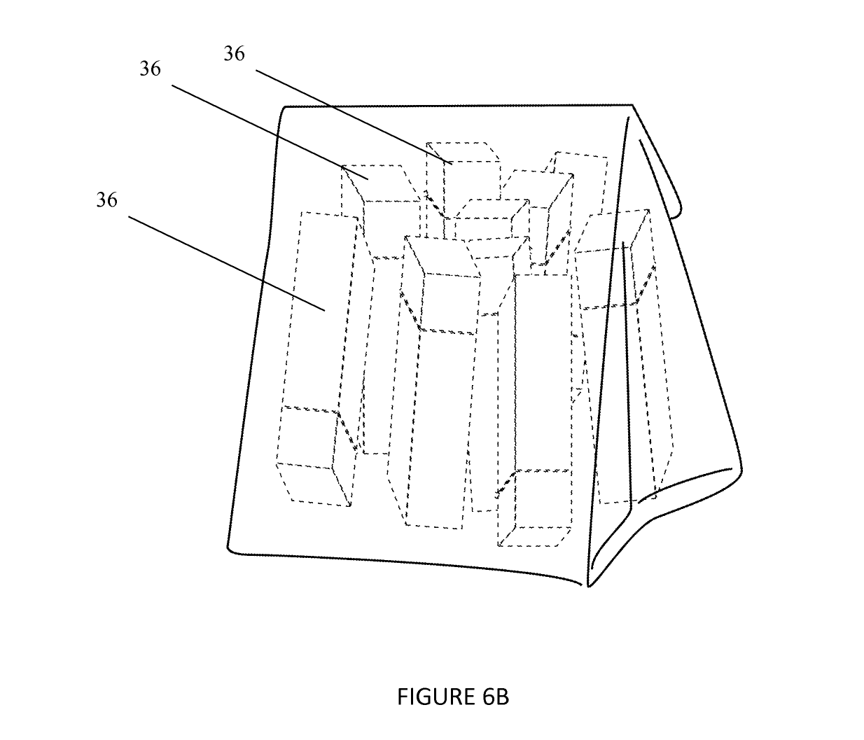

Referring now generally to the Figures, and particularly to FIG. 6A, FIG. 6A is a cut-away side view of a third container 36, containing a syringe 2. The third container 36, for the convenience of the user 16, contains a single syringe 2 which may be removed from the third container 36, used, and subsequently returned securely to the third container 36. Contained within the third container 36 may be either the first invented device 18, or the second invented device 26. The third container 36, having only one first invented device 18 or second invented cavity 36 contained therein, may be particularly convenient to a user 16, as the user 16 may not need a plurality of syringes 2 each time the user 16 goes out. As the third container 36 contains only a single syringe 2, the third container 36 may additionally be safely, easily and quickly disposed of by the user 16 upon completion of use. The third container 36 is shown in the Figure to have a quadrilateral cross-sectional shape, but may optionally be circular, polygonal or other suitable shape known in the art.

The materials used to create the third container 36 are preferably polyethylene, polyurethane, or polypropylene, but may be any other suitable material known in the art.

Referring now generally to the Figures, and particularly to FIG. 6B, FIG. 6B shows a plurality of the third containers 36 contained within a generic or prior art carrying case, such as a plastic or paper sack. FIG. 6B demonstrates the convenience of the third container 36, as a user may simply grab one of the third containers 36, and be easily on his or her way.

Referring now generally to the Figures, and particularly to FIGS. 7A through 7D, FIGS. 7A through 7D show a plurality of options for the configuration of the plunger projection set 20 and/or for the optional finger grip projection set 22. FIG. 7A shows two projections 38, which may lay on top of the plunger 8, creating a hermetic or near-hermetic seal; FIG. 7B shows a ring projection 40 through which a syringe 2 may pass in order to be securely stored within the first invented device 18 or the second invented device 26; FIG. 7C shows a single projection 38, which is sufficient to create a seal with the plunger 8; and FIG. 7D shows a plurality of spike projections 41, of which there may be as few or as many as is desired by the manufacturer. The projections 38, the ring projections 40, and/or the spike projections 41 given as examples in the Figures are to serve only as illustrative examples, and are not to be taken as limitations on the possibilities of means by which the syringe 2 may be secured within the first device 18 or the second invented device 26. For example, rather than a projection 38 or a ring projection 40, the entrance of the syringe 2 may trigger a spring-loaded reaction, such that, for example, a ball bearing may block the exit of the syringe 2 from the first device 18 or the second invented device 26. The ring projection 40 or the projection 38 may optionally be composed of any material known in the art sufficient to the task of creating a hermetic or near-hermetic seal when coupled with the plunger 8.

The plunger projection set 20 preferably protrudes toward the center of the first invented device 18 in a range of ratios of the diameter D1 of the plunger top 5. More particularly, the plunger projection set 20 preferably protrudes toward the first elongate cavity axis of the first cavity 18A of the first invented device 18 in a range of 0.1-0.5 of the diameter D1 of the plunger top 5. Yet more particularly, the plunger projection set 20 preferably protrudes toward the center of the first invented device 18, or the second invented device 26 at a ratio of 0.2-0.4 of the diameter D1 of the plunger top 5. The finger grip projection set 22, when present, preferably also protrudes toward the center of the first invented device 18 in a rage of ratios of the diameter D2 of the finger grip 6. More particularly, the optional finger grip projection set preferably protrudes toward the center of the first invented device 18 in a range of 0.1-0.5 of the diameter D2 of the finger grip 6. Yet more particularly, the finger grip projection set 21 preferably protrudes toward the center of the first invented device 18, or the second invented device 26 at a ratio of 0.2-0.4 of the diameter D2 of the finger grip 6.

Referring now generally to the Figures, and particularly to FIGS. 8A through 8E, FIGS. 8A through 8E show cut-away side views of a plurality of design variations for the invented devices. FIG. 8A shows the first invented device 18 as described in the above text, having the first device central axis length L5 along the first device elongate axis A2, a circular cross-sectional shape, and a consistent diameter D4 throughout the well 24. FIG. 8B shows the first invented device 18, surrounded by a casing 42 having a quadrilateral cross-sectional shape. FIG. 8C shows a third invented device 44, having a third body 44A that forms a first cavity 44B, and having a cylindrical top, where the plunger projection set 20 and the optional finger grip projection set 22 are placed, and a third well 46 having a quadrilateral length. The third well 46 further has a width W1 being narrower than the diameter D6 of the cylindrical portion, and third walls 47. FIG. 8D shows a fourth invented device 48, having a fourth body 48A that forms a fourth cavity 48B wherein a fourth well 50 is textured along the length of the fourth walls 51 for an increased security of fit with the barrel 8 of the syringe 2. FIG. 8E shows a fifth invented device 52, having a fifth body 52A that forms a fifth cavity 52B, wherein the entire fifth invented device 52 has an orthogonal shape, including the fifth well 54, and the portion of the fifth invented device 52 housing the plunger projection set 20 and the optional finger grip projection set 22. The fifth invented device 52 displays a consistent width W2 throughout the fifth well 54 and the portion of the fifth invented device 52 housing the plunger projection set 20 and the optional finger grip projection set 22.

The third invented device 44, the fourth invented device 48, and the fifth invented device 52 preferably form non-porous first bodies 44A, 48A, and 52A, respectively and may optionally be comprised of or including of any suitable water-resistant, non-porous materials known in the art, including, but not limited to polyethylene, polyurethane, or polypropylene, but may be any other suitable material known in the art.

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

Any of the steps, operations, or processes described herein may be performed or implemented with one or more hardware modules, alone or in combination with other devices.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based herein. Accordingly, the disclosure of the embodiments of the invention is intended to be illustrative, but not limiting, of the scope of the invention, which is set forth in the following claims.

### LIST OF ELEMENT NUMBERS

FIG. 1A
Syringe 2
Plunger 4
Plunger Top 5
Finger grip 6
Barrel 8
Needle 10
Diameter D1 of the plunger
Diameter D2 of the finger grip
Diameter D3 of the barrel
Length L1 of the plunger
Length L2 of the barrel
Length L3 of the needle
FIG. 1B
Plunger Cap 12
Needle Cap 14
User 16
Syringe length L4
First elongate syringe axis A1
FIG. 2A
First invented device 18
First body 18A that forms
First cavity 18B
Plunger projection set 20
Finger grip projection set 22
Well 24
First internal walls 25
Diameter D4 of the first well
First device length L5
First elongate device axis A2
FIG. 3A
Second invented device 26
Second body 26A
Second cavity 26B
Second well 28
Second internal walls 29
Second device length L6
Second elongate cavity axis A3
FIG. 4
First container 30
Unused syringe central axis length L7
FIG. 5
Second container 32
Slots 34
FIGS. 6A & 6B
Third container 36
FIGS. 7A-7D
Projection 38
Ring projection 40
Spike Projection 41
FIGS. 8A-8E
Square casing 42
Third invented device 44
Third well 46
Third body 44A
Third well 46
Third walls 47
Fourth invented device 48
Fourth body 48A
Fourth cavity 48B
Fourth well 50
Fourth walls 51
Fifth invented device 52
Fifth body 52A
Fifth cavity 52A
Width W1
Width W2

1. A device comprising:
a body comprising an internal wall coupled with a bottom well, the internal wall forming an elongate cavity (“cavity”); and
a first projection positioned within the cavity and extending from the internal wall, wherein the cavity is adapted to receive an entire syringe between the first projection and the bottom well, and the first projection is adapted to both permit an application of manual pressure to insert the entire syringe into the cavity and to inhibit removal of the syringe from the cavity.

2. The device of claim 1, wherein the first projection is a single protruding element extending from the internal wall and into the cavity.

3. The device of claim 1, wherein the first projection comprises a plurality of protruding elements, wherein each protruding element extends from the internal wall and into the cavity.

4. The device of claim 1, wherein the first projection comprises at least a single protruding partial ring element extending from the internal wall and into the cavity.

5. The device of claim 1, wherein the first projection is a single whole protruding ring element extending from the internal wall and into the cavity.

6. The device of claim 1, further comprising a second projection extending from the internal wall between the first projection and the bottom well and extending into the cavity.

7. The device of claim 6, wherein the second projection is a single secondary protruding element extending from the internal wall and into the cavity.

8. The device of claim 6, wherein the second projection comprises a plurality of secondary protruding elements, wherein each secondary protruding element extends from the internal wall and into the cavity.

9. The device of claim 6, wherein the comprises projection comprises at least a single secondary protruding partial ring element extending from the internal wall and into the cavity.

10. The device of claim 5, wherein the second projection is a single secondary whole protruding ring element extending from the internal wall and into the cavity.

11. The device of claim 1, wherein a cross-section of the cavity orthogonal to a central length axis of the cavity is substantively circular.

12. The device of claim 1, wherein a cross-section of the cavity orthogonal to a central length axis of the cavity is substantively quadrilateral.

13. The device of claim 1, wherein a cross-section of the cavity orthogonal to a central length axis of the cavity is substantively polygonal.

14. The device of claim 1, wherein the cavity comprises a first section having a first cross sectional shape orthogonal to a central length axis of the cavity and a second section having a second cross sectional shape orthogonal to the central length axis of the cavity.

15. The device of claim 14, wherein the first cross sectional shape is substantively polygonal.

16. The device of claim 14, wherein the first cross sectional shape is substantively circular.

17. The device of claim 16, wherein the second cross sectional shape is substantively polygonal.

18. The device of claim 16, wherein the first projection is positioned within the first section.

19. The device of claim 18, wherein the second projection is positioned within the first section.

20. The device of claim 19, wherein the first cross sectional shape is substantively circular.

21. A device comprising:
a plurality of syringe disposal containers, each disposal container comprising
a body comprising an internal wall and a bottom well, each internal wall forming an elongate cavity (“cavity”);
a first projection extending the internal wall and into the cavity, wherein the cavity is adapted to receive an entire syringe between the first projection and the bottom well, and the first projection is adapted to both permit an application of manual pressure to insert the entire syringe into the cavity and to inhibit removal of the syringe from the cavity; and
a frame, the frame adapted to substantively enclose the plurality of syringe disposal containers.

22. The device of claim 21, wherein at least one syringe disposal container is adapted to store a syringe with a finger grip positioned above the first projection of the at least one syringe container while a barrel of the syringe is substantively inserted into the cavity.

23. The device of claim 21, wherein each syringe container is adapted to store a syringe with a finger grip positioned above the first projection of the instant syringe container while a barrel of the syringe is substantively inserted into the cavity of the instant syringe container.

24. The device of claim 23, wherein the at least one syringe container further comprises a second projection positioned within the cavity of at least one syringe container between the first projection of the at least one syringe container and the bottom well of the at least one syringe container and extending into the cavity of the at least one syringe container.

25. The device of claim 23, the frame further comprising a syringe storage area, the syringe storage area adapted to store a plurality of syringes.

26. The device of claim 26, wherein the syringe storage area is adapted to store at least a number of syringes equal to the number of plurality of syringe containers.

27. A portable storage and disposal device comprising:
Means to accessibly store a first plurality of syringes; and
Disposal means to individually store each of a second plurality of used syringes (“disposal means”), the disposal means adapted to accept separate insertions of individual syringes and to inhibit access to each syringe after insertion.
